(19) United States
(12) Patent Application Publication
(10) Pub. No.: US 2012/0225991 A1
(43) Pub. Date: Sep. 6, 2012

(54) AMINO AND HYDROXYL FUNCTIONAL POLYESTERS

(75) Inventors: Mohamad Deeb SHALATI, LOUISVILLE, KY (US); Ajaya NANDA, LOUISVILLE, KY (US); Stephen HELLEMS, LOUISVILLE, KY (US)

(73) Assignee: NUPLEX RESINS B.V., BERGEN OP ZOOM (NL)

(21) Appl. No.: 13/472,570

(22) Filed: May 16, 2012

Related U.S. Application Data
(63) Continuation of application No. PCT/EP2010/067713, filed on Nov. 17, 2010.
(60) Provisional application No. 61/261,779, filed on Nov. 17, 2009.

(30) Foreign Application Priority Data
Nov. 27, 2009 (EP) 09177390.3

(51) Int. Cl. C07C 229/30; C09D 175/00; C08G 18/68

(52) U.S. Cl. 524/507; 560/170; 528/75; 524/874

(57) ABSTRACT

The invention relates to amino and hydroxy-functional polyesters, wherein the amine is in the form of aspartic acid esters functionality, and wherein the amino and hydroxy-functional polyester has (a) a molecular weight (Mn) of at least about 500, (b) an acid value of about 5 or less, (c) a hydroxyl value of about 30 or more, and (d) an amine value of about 30 or more, and (e) an amine functionality of less than 1.8. Preferably, the compound includes molecules having on the average: at least 1 secondary amino group as an aspartate, and/or at least 1 hydroxy group, and an average total functionality of about 1.8 or higher. More preferably, the molecular weight of the amino and hydroxy-functional polyesters is between 204 and 10,000 and preferably between 482 and 5000.

### CROSS REFERENCE TO RELATED APPLICATIONS

This application is a continuation of PCT application number PCT/EP2010/067713 filed on 17 Nov. 2010, which claims priority from U.S. provisional application No. 61/261,779 filed on 17 Nov. 2009, as well as from EP application number 09177390.3 filed on 27 Nov. 2009. All applications are hereby incorporated by reference in their entireties.

### BACKGROUND OF THE INVENTION

1. Field of the Invention

The present invention relates to amino and hydroxy-functional polyesters, to compositions comprising such polyesters; their use as binders or as modifiers in binder systems. These binders or binder systems are particularly suitable for crosslinking with polyisocyanate compounds to give crosslinked poly(urea/urethane) systems, which are useful as coatings, adhesives, sealants or caulking materials; either as one component composition, but preferably as two-component poly(urea/urethane) hybrid coating compositions. The present invention furthermore relates to said crosslinked systems and processes for manufacturing of the amino polyols.

2. Description of the Related Art

High performance, durable coatings based on acrylic and/or polyester polyols and isocyanates are well known in the automotive and industrial coating markets. However, the increased demands for lower VOC necessitate the need to lower the molecular weight of the polyols and/or the use of low molecular weight reactive diluents in order to increase solid contents of the coatings. As a result of incorporating these low molecular weight polyols, the speed of drying and early hardness development of coatings have suffered, in comparison with the conventional medium or low solids one- or two-component urethane coatings. The use of hindered secondary amine compounds along with isocyanate hardeners has alleviated, to some extent, the early cure problem due to the fast amine-NCO reaction, and the generation of harder and more polar urea groups in the resulting polyurea coatings.

Polyaspartate esters have been described in U.S. Pat. No. 5,126,170 and U.S. Pat. No. 5,236,741, which are hereby incorporated by reference in their entireties, for the preparation of a polyurea coating by coating the substrate with a coating composition containing a polyisocyanate component and an isocyanate-reactive component containing at least one polyaspartic acid ester and curing the composition to a temperature of 100° C. or less.

EP 1,038,897 A2, which is hereby incorporated by reference in its entirety, discloses the preparation of polyurea coatings comprising a polyisocyanate component, an isocyanate-reactive component, for coating the substrate with coating composition and curing at temperature less than 100° C. The isocyanate-reactive component comprises the reaction product of a diester of maleic or fumaric acid and polyamine; the polyamine having 2 primary amine groups and at least one other functional group which is reactive towards isocyanate at temperature below 100° C.

U.S. Pat. No. 5,596,044, which is hereby incorporated by reference in its entirety, discloses prepolymers derived from aminoalcohols, containing hydantoin group precursors and their use in coatings compositions.

U.S. Pat. No. 7,166,748, which is hereby incorporated by reference in its entirety, discloses a coating composition comprising a polyisocyanate, an isocyanate-reactive component having hydroxyl and aspartate functionality, coating a substrate with coatings composition containing amine and/or hydroxylamine compounds.

U.S. Pat. No. 5,633,389, which is hereby incorporated by reference in its entirety, discloses a thermoreversible process for the preparation of a hydantoin comprising the reaction of unsaturated polyester with mono-functional amine to yield poly(aspartate ester), reacting the aspartate ester with isocyanate to produce a poly(ester urea) and heating the poly(ester urea) to form a hydantoin compound.

U.S. Pat. No. 5,561,214, which is hereby incorporated by reference in its entirety, describes the composition and process for making hyperbranched polyaspartate esters.

U.S. Patent Application 2005/0059792 A1, which is hereby incorporated by reference in its entirety, describes a method for preparing flexible polyaspartate esters by the incorporation of unsaturated oligoester prepared by the transesterification of α,β-unsaturated esters with hydroxyl-functional compounds and reacting the transesterified product with primary di-amine and reacting the remaining primary amine groups with α,β-unsaturated esters.

The prior art, EP0604814 (Canadian Pat. Appl. No. 2,111,927), which is hereby incorporated by reference in its entirety, describes a process for the production of amino polyester resins derived from the reaction of unsaturated polyester resins and monoamine. The unsaturated polyester resins contain at least two maleate or fumarate units of the following structure:

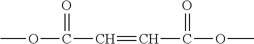

The nitrogen weight percent (wt. %) is between 0.01 to 9.0% and hydroxyl weight percent (% OH) is between 0-10%.

Although the prior art uses aspartate esters in the manufacture of coatings, a further improvement in properties with respect to flexibility, adhesion, durability, combined with balanced cure speed remains a challenge.

### BRIEF SUMMARY OF THE INVENTION

It is an object of the invention to provide amino and hydroxy-functional polyesters, to compositions comprising such polyesters for use as binders or as modifiers in binder systems. These binders or binder systems are particularly suitable for crosslinking with polyisocyanate compounds to give crosslinked poly(urea/urethane) systems, which are useful as coatings, adhesives, sealants or caulking materials; either as one component composition, but preferably as two-component poly(urea/urethane) hybrid coating compositions. These crosslinked systems and processes for manufacturing of amino polyols.

In a first embodiment, the present invention relates to amino and hydroxy-functional polyesters, wherein the amino functionality is a secondary amine in the form of aspartic acid esters, and wherein the amino and hydroxy-functional polyesters have
(a) a molecular weight (Mn) of at least about 500
(b) an acid value of about 5 or less
(c) a hydroxyl value of about 30 or more
(d) an amine value of about 30 or more
(e) the hydroxyl is a sterically-hindered primary and/or secondary hydroxyl.
   Preferably, the amino and hydroxy-functional molecules of the polyester have on average
(f) at least 1 secondary amine group as an aspartate ester,
(g) and/or at least 1 hydroxyl group,
(h) an average total functionality of secondary amine and hydroxyl group of 1.8 or more per molecule.

In a second embodiment of the invention, the invention relates to low molecular weight amino and hydroxy-functional polyesters which are derived from the reaction of unsaturated oligoesters and mono primary amine. The unsaturated polyesters have on the average at least one and a maximum of 1.8 fumarate or maleate units. The amino functionality is between 0.8 and 1.8 groups per molecule which is the form of a secondary amine as aspartic acid ester. The hydroxyl functionality has a minimum value of 1 and a maximum of 12 hydroxyl groups per molecule. The number average molecular weight of the amino and hydroxy-functional polyesters is between 204 and 10,000 and more preferably between 482 and 5000. The amino and hydroxy-functional polyesters contain 0.1 to 7% by weight nitrogen in the form of secondary amine as aspartate groups and 0.1% to 10% of primary and/or secondary hydroxyl groups. The preferred OH groups are a secondary and/or hindered primary hydroxyl groups. The maximum acid value of the amino and hydroxy-functional polyesters is 2 and preferably less than 1.

The invention furthermore relates to curable compositions comprising said amino and hydroxy-functional polyester and a polyisocyanate, wherein the amount of isocyanate is present in about 60% of the molar amount of the amine and alcohol groups, or more.

In a preferred embodiment, the amino and hydroxy-functional polyesters comprise either one or a mixture of the following compounds:

Amino and Hydroxy-functional Polyester (Formula I);

Another preferred embodiment of the present invention comprises a blend of the above amino and hydroxy-functional polyester with any of the following commercially available components:

1) Hydroxy-functional polyesters
2) Hydroxy-functional acrylic polymers
3) Other hydroxy-functional polymers
4) Polyaspartic acid esters

In one preferred embodiment, the amino and hydroxy-functional polyester has the general structure according to Formula I:

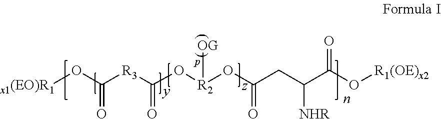

Wherein: R=mono-valent alkyl, aryl and/or arylalkyl radical
R₁=residue obtained from a polyol after removing the OH groups and having a valency of 1 to 6
R₂=residue obtained from a polyol after removing the OH groups and having a valency of 2 to 6
R₃=divalent saturated and/or unsaturated alkyl and/or aryl radical
E=H or acyl group having 1 to 18 carbon atoms
X¹, X²=an integer having an equal or different values of 0 to 5, but and the sum of X¹ and X² is at least 1
y, z=an integer having a value of 0 or 1
p=an integer having a value between 0 to 4
G=E and/or is a residue having the following structure:

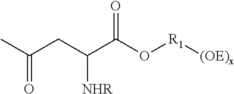

n=an integer having a value between 1 to 10.
The number average molecular weight of the amino and hydroxy-functional polyester in Formula I preferably is about 500 or higher and about 5,000 or lower in the above embodiment. Further, the polydispersity of the amino and hydroxy-functional compound is about 4 or lower, and about 1.2 or higher.

Additionally, in one embodiment, in Formula I, the amino and hydroxy-functional polyester has a hydroxyl value of about 40 or higher and of about 300 or lower.

In another embodiment, in Formula I, the amino and hydroxy-functional polyester has an amine value of about 40 or higher and of about 300 or lower.

In yet another embodiment, in Formula I, the amino and hydroxy-functional polyester has an average total functionality of about 1.8 or more and of about 10 or less.

In a further preferred embodiment, the general structure of the amino and hydroxy-functional polyesters is shown in Formula II.

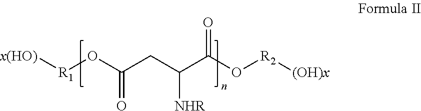

Wherein: R=mono-valent alkyl, aryl and/or arylalkyl radical and may contain OH group R₁ and R₂ may be similar or different and each is a residue obtained from a polyol after removing the OH groups and having a valency of 1 to 6, and x=is an integer having a value between 0 to 6, and n has a value between 1 to 1.8.

In yet another preferred embodiment of the present invention, an amino and hydroxy-functional polyester is prepared by a method comprising the preparation of an unsaturated hydroxyl functional polyester comprising at least one of: maleate and fumarate unsaturation, and wherein the maleate and fumarate unsaturation is reacted with an aliphatic or aromatic amine compound to prepare an aspartate through a pseudo Michael addition reaction.

Pseudo Michael Addition reaction is defined here as the addition of the primary amine, as the Michael Addition donor, to the unsaturated polyester double bond, as the Michael Addition acceptor.

In yet another preferred embodiment of the present invention, a coating composition comprises the following components in parts by weight (pbw) is prepared from:

(a) aminopolyol of the invention (1-80 pbw)
(b) polyisocyanate compound (1-65 pbw)
(c) other binder constituents (0-60 pbw)
(d) colorants (0-40 pbw)
(e) additives (0-10 pbw)
(f) catalysts (0-1 pbw)
(g) solvents (0-30 pbw)
In which components a-f together are 100.

In a further embodiment based on the above, component (c) is present in an amount between 1 and 60 pbw, and wherein component (c) comprises one or more of:
i. hydroxy functional acrylic polymers, hydroxy functional polyester, hydroxy functional reactive diluent, hydroxy functional polyether, hydroxy functional polycarbonate or hydroxy functional polyurethane;
ii. non-functional or lightly functional polymers with a functionality; equivalent weight of about 5000 or higher; and
iii. aspartate functional compounds other than compound (a).

In further embodiments, component (c) is present in an amount between 5 and 50 pbw, and/or comprises a hydroxy functional acrylic polymer, and/or comprises an aspartate functional compound other than component (a). Further, in another embodiment component (g) is present in about 10 pbw relative to components (a) through (f) or less. The novel amino and hydroxy-functional polyester of the present invention appears to be very suitable as binders for crosslinking with polyisocyanates to give fast curing poly(urea/urethane) hybrid coatings having good flexibility, durability, chemical resistance and low VOC. None of the prior art teaches the preparation of polyesters having both aspartate and hydroxyl functional groups as defined, which are useful for the preparation of poly(urea/urethane) hybrid coatings in a coatings composition comprising aspartate and hydroxyl functionality with polyisocyanate hardeners.

### BRIEF DESCRIPTION OF THE DRAWINGS

The features and advantages of the invention will be appreciated upon reference to the following drawings, in which:

FIG. 1 shows the 20° Gloss Retention of White paints based on Amino and Hydroxy-Functional Polyesters at various QUV 313 exposure time according to the invention and as described in several of the examples.

### DESCRIPTION OF ILLUSTRATIVE EMBODIMENTS

The following is a description of certain embodiments of the invention, given by way of example only and with reference to the drawings. The present invention relates to amino and hydroxy-functional polyesters, wherein the amine is a secondary amine in the form of aspartic acid esters, and wherein the amino and hydroxy-functional compounds have (a) a molecular weight (Mn) of at least about 500, (b) an acid value of about 5 or less, (c) a hydroxyl value of about 30 or more, (d) an amine value of about 30 or more, and an amine functionality lower than 1.8.

Preferably, the amino and hydroxy-functional molecules have, on average, at least about 1 secondary amine group in the form of aspartic acid esters and at least about 1 hydroxyl group, and the average total amino and hydroxy functionality is at least about 1.8 or more per molecule.

The number average molecular weight of the amino and hydroxy-functional polyester is about 500 or higher, preferably about 600 or higher. In a preferred embodiment, the number average molecular weight of the amino and hydroxy-functional polyesters is between 204 and 10,000 and more preferably between 482 and 5000. Generally, the number average molecular weight (Mn) is about 5,000 or lower, preferably about 3,000 or lower and even more preferred about 1200 or lower. The number average molecular weight may be for example in the range of 500-3000 or 600 to 5000. The molecular weight is expressed in Dalton, and the molecular weight and polydispersity are measured by GPC against polystyrene standard in THF; with a column adapted for measuring the appropriate molecular weight.

The polydispersity of the amino and hydroxy-functional polyester preferably is about 4 or lower, more preferably about 2.5 or lower. Generally, the polydispersity will be about 1.2 or higher. A lower polydispersity has the advantage of improving the viscosity such that less organic solvent is required.

The acid value of the amino and hydroxy-functional polyester is generally about 5 or less, and even more preferable about 3 or less, like for example between 0 and 3.

The amino and hydroxy-functional polyester generally has a hydroxyl value of about 30 or higher, preferably of about 40 or higher. Generally, the hydroxyl value will be about 300 or lower, preferably about 200 or lower, and most preferable about 150 or lower. A high hydroxyl value may cause brittleness because of a too high crosslinking density. The hydroxyl value of about 30 or higher is important to achieve a stable coating with good properties. It will be appreciated that the amine value is expressed in the weight of KOH in milligrams that is equal in basicity to NH present in 1 gram compound. The hydroxyl value is also expressed in number of milligrams of KOH that will neutralize the acetic acid liberated from the reaction of an OH-functional compound with acetic acid anhydride.

The amino and hydroxy-functional polyester generally has an amine value of about 30 or higher, preferably an amine value of about 40 or higher. Generally, the amine value will be about 300 or lower, preferably about 200 or lower. Most preferred is an amine value between 50 and 150. The amino-functionality is an aspartate-type functionality.

The amino and hydroxy-functional polyester preferably has an average functionality of about 1.8 or more, preferably about 2.0 or more, and more preferable about 2.5 or more, and even more preferably about 3 or more. Generally, the average functionality will be about 10 or less, preferably about 5 or less. The functionality may be for example in the range between 2 and 5, or 2 and 10.

The hydroxyl equivalent weight preferably is about 2500 or lower and more preferably about 2000 or lower. Generally, the hydroxy equivalent weight will be about 300 or higher, preferably about 400 or higher, and more preferably about 500 or higher. The equivalent weight may be for example between 400 and 2000.

The amine equivalent weight preferably is about 2000 or lower. Generally, the amine equivalent weight will be about 1500 or lower, preferably about 1000 or lower, and more preferably about 600 or lower. The amine equivalent weight preferably will be about 150 or higher, preferably about 200 or higher. The equivalent weight may be for example between 150 and 1000, or 200 and 1500.

The total hydroxyl and amine value can be measured in standard ways, wherein the hydroxyl and amine groups are reacted with an excess of acetic acid anhydride, and the resulting free acetic acid group is back titrated with KOH to assess the total millimolar amount of hydroxy and amine groups in 1 gram of sample. The amine value can also be assessed by titration with 0.1 N hydrochloric acid (ASTM D2572-91) and thereafter calculated back to mg KOH. The hydroxyl value can be calculated by their theoretical molar amount by subtracting the amine value from the total amine and hydroxyl value.

The amino and hydroxy-functional polyester can be made in a plurality of ways.

In a preferred process for the preparation of a polyester based amino and hydroxy-functional compounds, an unsaturated hydroxyl functional polyester is prepared (in one or more synthetic steps), which comprises maleate or fumarate unsaturation. The maleate and/or fumarate unsaturation is used to prepare an aspartate through addition of an aliphatic or an aromatic primary amine compound.

The invention furthermore relates to curable compositions comprising said amino and hydroxy-functional polyester and polyisocyanates, wherein the amount of isocyanate is present in about 60% of the molar amount of the amino and alcohol groups (isocyanate reactive group), or more.

Preferably, the amount of isocyanate is about 70% of the molar amount of the amino groups and alcohol groups or more, and even more preferable about 80% or more. Generally, the amount is about 140% or less, preferably 110% or less. The most preferred amount is about the same amount of isocyanate and isocyanate reactive groups.

The coating composition contains at least the amino and hydroxy-functional component of the invention and an isocyanate compound. However, several other components may be present, such as (a) other binder components, (b) non-reactive diluents, (c) coloring agents, (d) catalysts, flow agents, and other commonly used additives.

The other binder components may be further polymers, reactive diluents and non-reactive polymers, where introducing the latter compounds would result in the formation of an interpenetrating network (IPN). Suitable other binder polymeric additives comprise polyester polyols, polyacrylic polyols, polyether polyols and the like. Aspartate ester functional components other than described in the present invention, but for example described in the prior art may be used in admixture with the amino and hydroxy-functional compounds of the present invention.

As non-reactive diluents (or solvents), the conventional organic solvents can be used. The coating composition can be formulated as a high solid coating with relatively low amount of solvents and/or other volatile organic compounds (VOCs). Generally, the amount of VOCs is about 20 parts by weight (pbw) of the total coating composition or less, preferably about 10 pbw or less and more preferably is about 5 pbw or less.

As coloring agents, the conventional pigments, extenders, and dyes can be used, preferably in a pigment dispersion in which the pigment is stabilized for dispersion into an organic coating composition. Suitable pigments include organic and inorganic pigments. The inorganic pigments include titanium dioxide, iron oxides, zinc oxide, other metal oxides and carbon black. The organic pigments include phthalocyanine blue and green pigments, perylenes, pyrrole, arylides, indanthrones, magenta, and quinacridone red, and many other pigments. The color pigment may be chosen from those disclosed by HERBST et al., Industrial Organic Pigments, Production, Properties, Applications; 3rd Edition, Wiley-VCH, 2004, ISBN 3527305769, which is hereby incorporated by reference in its entirety. Suitable extenders include calcium carbonate, talc, barium sulfate, hydrated aluminum silicate (Pyrophyllite), calcium metasilicate (Wollastonites), kaolin clays, and other fillers.

Suitable additives comprise catalysts [such as for example Tin (IV) compounds] thixotropes, defoamers, pigment dispersants, flow agents, extenders, dehydrating agents (like molecular sieves) and the like.

The coating composition generally comprises the following components in parts by weight (pbw)

(a) aminopolyol of the invention (1-80 pbw)
(b) polyisocyanate compound (1-65 pbw)
(c) other binder constituents (0-60 pbw)
(d) colorants (0-40 pbw)
(e) additives (0-10 pbw)
(f) catalysts (0-1 pbw)
(g) solvents (0-30 pbw)
In which components a-f together are 100.

In a further preferred embodiment, component (c) is present in an amount between 1 and 60 pbw, preferably between 40 and 60 pbw.

In a further preferred embodiment, the invention provides a high-solid coating composition, wherein component (g) is present in about 10 pbw relative to components (a) through (f) or less.

In general, the hydroxy-groups in the amino and hydroxy-functional polyester having the least steric hindrance around the OH group reacts faster with isocyanate to form urethane linkage than sterically hindered OH group. Thus, primary hydroxyl groups react with NCO faster than secondary and even much faster than tertiary hydroxyl groups. In a preferred embodiment, the polyester comprises secondary hydroxyl and/or sterically hindered primary hydroxyl groups as reactive groups for the reaction with isocyanates. A sterically hindered primary hydroxyl groups are those with substituents (X1, X2) at the 2-position as shown in the following structure:

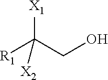

Preferably the hydroxy-functional polyester will have a number average molecular weight of at least about 100. Typical number average molecular weights will range from about 100 to about 10,000, and especially 100 to about 3,000. In order to control the duration of the pot-life of the final 2-component coatings, and thus the rate of viscosity increase, it is preferred in the practice of this invention to utilize hydroxy-functional polyesters having either primary and/or secondary and even tertiary hydroxyl functionality. The more the steric hindrance of the hydroxyl group the slower the rate of cure will be.

Representative hydroxy-functional polyesters include those described below: Hydroxy-functional polyesters are those prepared by condensation polymerization reaction techniques are well known in the art. Representative condensation polymerization reactions include polyesters prepared by the condensation of polyhydric alcohols and polycarboxylic acids or anhydrides, with or without the inclusion of drying oil, semi-drying oil, or non-drying oil fatty acids. By adjusting the stoichiometry of the alcohols and the acids while maintaining an excess of hydroxyl groups, hydroxy-functional polyesters can be readily produced to provide a wide range of desired molecular weights, unsaturation content and performance characteristics.

The polyester polyols are derived from one or more aromatic and/or aliphatic polycarboxylic acids, the anhydrides thereof, and one or more aliphatic and/or aromatic polyols. The carboxylic acids include the saturated and unsaturated polycarboxylic acids and the derivatives thereof, such as maleic acid, fumaric acid, succinic acid, adipic acid, azelaic acid, dicyclopentadiene dicarboxylic acid, hexahydrophthalic anhydride, methyl-hexahydrophthalic anhydride, aromatic polycarboxylic acids, such as phthalic acid, isophthalic acid, terephthalic acid, etc Anhydrides such as maleic anhydride, phthalic anhydride, trimellitic anhydride, or Nadic Methyl Anhydride (brand name for methylbicyclo[2.2.]heptene-2,3-dicarboxylic anhydride isomers) can also be used.

Representative saturated and unsaturated polyols which can be reacted in stoichiometric excess with the carboxylic acids to produce hydroxy-functional polyesters include diols such as ethylene glycol, dipropylene glycol, 2,2,4-trimethyl 1,3-pentanediol, neopentyl glycol, 1,2-propanediol, 1,4-butanediol, 1,3-butanediol, 2,3-butanediol, 1,5-pentanediol, 1,6-hexanediol, 2,2-dimethyl-1,3-propanediol, 1,4-cyclohexanedimethanol, 1,2-cyclohexanedimethanol, 1,3-cyclohexanedimethanol, 1,4-bis(2-hydroxyethoxy)cyclohexane, trimethylene glycol, tetramethylene glycol, pentamethylene glycol, hexamethylene glycol, decamethylene glycol, diethylene glycol, triethylene glycol, tetraethylene glycol, norbornylene glycol, 1,4-benzenedimethanol, 1,4-benzenediethanol, 2,4-dimethyl-2-ethylenehexane-1,3-diol, 2-butene-1,4-diol, and polyols such as trimethylolethane, trimethylolpropane, trimethylolhexane, triethylolpropane, 1,2,4-butanetriol, glycerol, pentaerythritol, dipentaerythritol, etc.

Typically, the reaction between the polyols and the polycarboxylic acids is conducted at about 120° C. to about 220° C. in the presence or absence of an esterification catalyst such as dibutyl tin oxide.

Additionally, hydroxy-functional polyesters can be prepared by substituting some or all of the polyols described above with epoxides and/or polyepoxides where acids and anhydride can open the oxirane ring to form the corresponding ester and hydroxy groups. Representative polyepoxides include those prepared by condensing a polyhydric alcohol or polyhydric phenol with an epihalohydrin, such as epichlorohydrin, usually under alkaline conditions. Some of these condensation products are available commercially under the designations EPON or DER from Hexion Specialty Chemicals or Dow Chemical Company, respectively, and methods of preparation are representatively taught in U.S. Pat. Nos. 2,592,560; 2,582,985 and 2,694,694 all of which are incorporated by reference in their entirety.

If epoxy compounds are used during the preparation of hydroxy-functional polyesters, cycloaliphatic epoxies are the preferred epoxies. Commercial examples of representative preferred cycloaliphatic epoxies include 3,4-epoxycyclohexylmethyl 3,4-epoxycyclohexane carboxylate (e.g. “ERL-4221” from Dow Chemical); bis(3,4-epoxycyclohexylmethyl)adipate (e.g. “ERL-4299” from Dow Chemical); 3,4-epoxy-6-methylcyclohexylmethyl 3,4-epoxy-6-methylcyclohexane carboxylate (e.g. “ERL-4201” from Dow Chemical); bis(3,4-epoxy-6-methylcyclohexylmethyl)adipate (e.g. “ERL-4289” from Dow Chemical); bis(2,3-epoxycyclopentyl)ether (e.g. “ERL-0400” from Dow Chemical); dipentene dioxide (e.g. “ERL-4269” from Dow Chemical); 2-(3,4-epoxycyclohexyl-5,5-spiro-3,4-epoxy)cyclohexane-metadioxane (e.g. “ERL-4234” from Dow Chemical). Other commercially available cycloaliphatic epoxies are available from Ciba-Geigy Corporation such as CY 192, a cycloaliphatic diglycidyl ester epoxy resin having an epoxy equivalent weight of about 154. The manufacture of representative cycloaliphatic epoxies is taught in various patents including U.S. Pat. Nos. 2,884,408, 3,027,357 and 3,247,144 all of which are incorporated by reference in their entirety.

Other polyepoxides potentially useful in the practices of this invention include aliphatic and aromatic polyepoxies, such as those prepared by the reaction of an aliphatic polyol or polyhydric phenol and an epihalohydrin. Other useful epoxies include epoxidized oils and acrylic polymers derived from ethylenically unsaturated epoxy-functional monomers such as glycidyl acrylate or glycidyl methacrylate in combination with other copolymerizable monomers such as those listed below.

Another method to form particularly preferred hydroxy-functional polyesters comprises chain extending the hydroxyl-functional polyesters by reacting the hydroxyl groups of a (precondensed) polyester with chain extenders, preferably polyalkylene oxide or lactones such as polyethylene oxide, polypropylene oxide or caprolactone, valerolactone, and butyrolactone.

Monocarboxylic acids can be used for the preparation of hydroxy-functional polyesters to control molecular weight, functionality, and other characteristic properties. The monocarboxylic acids can be aliphatic, cycloaliphatic, aromatic or mixtures thereof. Preferably, the monocarboxylic acid contains 6 to 18 carbon atoms, most preferably 7 to 14 carbon atoms, such as octanoic acid, 2-ethylhexanoic acid, isononanoic acid, decanoic acid, dodecanoic acid, benzoic acid, hexahydrobenzoic acid, and mixtures thereof.

Monohydroxy compounds can be used in the practice of this invention to control molecular weight, functionality, and other characteristic properties. Examples of suitable monofunctional alcohols include alcohols with 4-18 carbon atoms such as 2-ethyl butanol, pentanol, hexanol, dodecanol, cyclohexanol and trimethyl cyclohexanol.

Hydroxy-functional acids can be used to replace some and/or all of the acids and polyols described above. Typical hydroxy acids that can be used include dimethylol propionic acid, hydroxypivalic acid, and hydroxystearic acid.

In a further preferred embodiment according to the invention, the amino and hydroxy-functional polyesters are prepared from the addition reaction of primary amines to hydroxy-functional unsaturated polyester at temperatures ranging from 0° C. to 80° C. The hydroxy-functional unsaturated polyesters are prepared from either the polycondensation of polyols and polycarboxylic acid containing maleic anhydride and/or fumaric acid or from the addition reaction of monoalkyl maleate to epoxy compounds. The ratio of the hydroxyl groups to the carboxylic acid groups in the hydroxyl functional polyester is always higher than 1:1 and it can range from 1.1 to 3:1.

In general, the reaction of primary amines with maleate or fumarate esters produce aspartate groups with different degree of reactivity toward NCO. The higher the steric hindrance around the nitrogen atom of the amine, the slower the reactivity toward NCO and visa versa.

Primary amines useful for the present invention include, but not limited to, various alkyl, aryl or aralkyl amines having 1-30 carbon atoms in the molecule. Specific examples for the alkyl amine include methylamine, ethylamine, propyl- and isopropy; amine, butyl-isobutyl- and teriarybutylamine, 1,3-dimethyl- and 3,3-dimethylbutylamine, pentyl-isopentyl-tertiaryamy, and neopentylamine, hexylamine isomers, cyclohexylamine, 2-methylcyclohexylamine isomers, 4-methylcyclohexylamine isomers, cycloheptylamine, heptylamine isomers, octylamine isomers, nonylamine, dodecvylamine, stearylamine, cyclohexylmethylamine, α-methylcyclohexanemethylamine. Examples of aryl amines include aniline, toluidine isomers, aniline, dimethylaniline isomers, ethylaniline isomers, propyl- and isopropylaniline isomers, 2,6-diethylaniline, and various substituted anilines. Examples of aralakyl amines include benzyl amine, α-methylbenzylamine, α-ethylbenzylamine, 4, α-dimethylbenzylamine, phenethylamine, alkyl phenethylamine isomers, and 4-phenylbutylamine. Other amines suitable for the present invention are amino polyols such as 1-aminohydroxyprpoane isomers, 2-amino-2-methyl-1,3-propanediol, 2-amino-2-ethyl-1,3-propanediol, 2-amino-2-hydroxymethyl-1,3-propanediol, and alkyl esters of amino acids such as methyl, ethyl, propyl and butyl esters of glycine, alanine, phenylalanine, leucine, isoleucine, aspartic acid, glutamic acid, and valine.

The coating composition of the invention comprises (optionally blocked) isocyanate-functional cross-linkers. These compounds are based on the usual isocyanate-functional compounds known to a person skilled in the art. More preferably, the coating composition comprises cross-linkers with at least two isocyanate groups. Examples of compounds comprising at least two isocyanate groups are aliphatic, alicyclic, and aromatic isocyanates such as hexamethylene diisocyanate, 2,4,4-trimethyl hexamethylene diisocyanate, dimeric acid diisocyanate, such as DDI® 1410 ex Henkel, 1,2-cyclohexylene diisocyanate, 1,4-cyclohexylene diisocyanate, 4,4′-dicyclohexylene diisocyanate methane, 3,3′-dimethyl-4,4′-dicyclohexylene diisocyanate methane, norbornane diisocyanate, m- and p-phenylene diisocyanate, 1,3- and 1,4-bis(isocyanate methyl)benzene, 1,5-dimethyl-2,4-bis(isocyanate methyl)benzene, 2,4- and 2,6-toluene diisocyanate, 2,4,6-toluene triisocyanate, α,α,α′,α′-tetramethyl o-, m-, and p-xylylene diisocyanate, 4,4′-diphenylene diisocyanate methane, 4,4′-diphenylene diisocyanate, naphthalene-1,5-diisocyanate, isophorone diisocyanate, 4-isocyanatomethyl-1,8-octamethylene diisocyanate, the isocyanates described for the preparation of hydroxyfunctional urethanes above, and mixtures of the aforementioned polyisocyanates.

Other (optionally blocked) isocyanate compounds are based on adducts of polyisocyanates, e.g., biurets, isocyanurates, imino-oxadiazinediones, allophanates, uretdiones, and mixtures thereof. Examples of such adducts are the adduct of two molecules of hexamethylene diisocyanate or isophorone diisocyanate to a diol such as ethylene glycol, the adduct of 3 molecules of hexamethylene diisocyanate to 1 molecule of water, the adduct of 1 molecule of trimethylol propane to 3 molecules of isophorone diisocyanate, the adduct of 1 molecule of pentaerythritol to 4 molecules of toluene diisocyanate, the isocyanurate of hexamethylene diisocyanate, available from Bayer under the trade designation Desmodur® N3390, a mixture of the uretdione and the isocyanurate of hexamethylene diisocyanate, available from Bayer under the trade designation Desmodur® N3400, the allophanate of hexamethylene diisocyanate, available from Bayer under the trade designation Desmodur® LS 2101, and the isocyanurate of isophorone diisocyanate, available from Evonik, under the trade designation Vestanat® T1890. Furthermore, (co)polymers of isocyanate-functional monomers such as α,α′-dimethyl-m-isopropenyl benzyl isocyanate are suitable for use. Finally, as is known to the skilled person, the above-mentioned isocyanates and adducts thereof may be at least partly present in the form of blocked isocyanates.

For blocking the polyisocyanates it is possible in principle to employ any blocking agent which can be employed for the blocking of polyisocyanates and has a sufficiently low deblocking temperature. Blocking agents of this kind are well known to the skilled worker and need not be elucidated further here. It is possible to employ a mixture of blocked polyisocyanates which contains both isocyanate groups blocked with a first blocking agent and isocyanate groups blocked with a second blocking agent. Reference is made to WO 98/40442, which is hereby incorporated by reference in its entirety.

All patent applications and patents cited herein are herein incorporated by reference.

The invention will be further elucidated with the following non-limiting examples:

### EXAMPLES

### A) Preparation of Amino Polyester Polyols

Example 1 is an example of for the preparation of polyester with on average 1.2 hydroxyl groups and 1.2 NH groups per molecule.

### Example 1-A

### Preparation of Unsaturated Polyester Polyols

A four-necked reaction flask equipped with a condenser, agitator, heating mantle; addition funnel, thermocouple attached to a temperature control, and Dean-Stark trap primed with xylene, is charged with 44.34 parts (by weight) of neopentyl glycol (NPG), 5.02 parts of trimethylol propane (TMP), 18.95 of adipic acid (AdA), 17.78 maleic anhydride (MAn), 24.64 parts of isononanoic acid (iNA), 0.14 part of triphenyl phosphate, and, 0.035 part of Fascat 9100 (butyl stanoic acid catalyst available from Arkema Inc.), and heated under 0.5 SCFH (standard cubic feet per hour) (0.014 m³ hr⁻¹) nitrogen flow to 195-200° C. At 165° C., water starts to distil azeotropically. The reaction temperature is gradually increased to 200° C. and maintained at such temperature until an acid value of less than 2 is attained. The azeotropic water collected is 10.7 parts. The Dean-Stark trap is drained and the reaction mixture is purged with nitrogen to remove most of the volatiles. The unsaturated polyester polyol (UPPO) is cooled to 150° C. and filtered.

The obtained UPPO has a non-volatile material of 96.45% (measured by mixing the resin with NCO at NCO/OH of 1:1 and heated for 1 hr. at 130° C.); an acid value of 1.25; a hydroxyl value of 103; a number average molecular weight (Mn) of 1013; a weight average molecular weight (Mw) of 1900; and a polydispersity of 1.87.

### Example 1-B

### Preparation of Amino Polyester Polyol (APPO)

A four-neck reaction flask equipped as in Example 1-A, is charged with 82.2 parts of UPPO from Example 1-A, and heated to 40° C. α-Methylbenzylamine (17.8 parts) is added drop-wise via addition funnel over 1 hour while maintaining the temperature at or below 50° C. Heating is continued overnight at 50° C. The resultant light yellow viscous liquid obtained is filtered. The resulting resin has a non-volatile material (NVM) of 97.2%; a viscosity of 8000 m Pa·s; a number average molecular weight (Mn) of 1070; a weight average molecular weight (Mw) of 1950; and a polydispersity of 1.82. The hydroxyl equivalent weight is 658 (OH value=85), and the amine equivalent weight is 669 (amine value=84) and the average equivalent weight for the total functionality is 332. The amine value is determined by titrating the amine with 0.1 N hydrochloric acid (ASTM method D2572-91). The total hydroxyl plus the amine value is determined by acetylating both the hydroxy and amine groups with acetic acid anhydride and then titrating acetic acid with KOH. The hydroxyl value is then determined by subtracting the amine value from the total amine plus hydroxyl values.

### Example 2

Example 2 is an example of for the preparation of polyester with on average 1 hydroxyl groups and 4 NH groups per molecule.

### Example 2-A

### Preparation of Unsaturated Polyester Polyol

The procedure of Example 1-A is used for this example. Thus from 45.25 parts of neopentyl glycol (NPG), 5.69 parts of trimethylol propane (TMP), 37.35 maleic anhydride (MAn), 20.95 parts isononanoic acid, 0.14 part of triphenyl phosphate, and 0.05 part of Fascat 9100, an unsaturated polyester polyol (UPPO) is obtained. This UPPO has a non-volatile material of 99% (measured by mixing the resin with NCO at NCO/OH of 1:1 and heated for 1 hr. at 130° C.); an acid value of 1.0; a hydroxyl value of 125; a number average molecular weight (Mn) of 1150; a weight average molecular weight (Mw) of 1800; and a polydispersity of 1.6.

### Example 2-B

### Preparation of Amino Polyester Polyol (APPO)

The procedure of Example 1-B is used for this example. Thus from 69.1 parts of UPPO from Example 2-A, and 30.9 parts α-Methylbenzylamine a viscous yellow resin of amino polyester polyol (APPO) is obtained. The APPO has a non-volatile material (NVM) content of 99.75%; a viscosity of 435 m Pa·s; a Mn of 1200; a Mw of 1900; and a polydispersity of 1.6. This APPO has a hydroxyl equivalent weight of 1452, an amine equivalent weight of 380 and an average equivalent weight for the total functionality of 301.

### Example 3

Example 3 is an example of the preparation of polyester with on average 1 hydroxyl groups and 2 NH groups per molecule.

### Example 3-A

### Preparation of Unsaturated Polyester Polyol

The procedure of Example 1-A is used for this example. Thus from 44.17 parts of neopentyl glycol (NPG), 5.02 parts of trimethylol propane (TMP), 11.46 parts of adipic acid (AdA), 25.00 maleic anhydride (MAn), 24.55 parts isononanic acid, 0.14 part of tirphenyl phosphate, and 0.05 part of Fascat 9100, a viscous UPPO resin is obtained. The unsaturated polyester polyol (UPPO) is cooled to 150° C. and filtered.

The resulting UPPO has a non-volatile material of 99%, an acid value of 1.0; a hydroxyl value of 125; a number average molecular weight (Mn) of 1177; a weight average molecular weight (Mw) of 2800, a hydroxyl equivalent weight of 732, an unsaturation equivalent weight of 392.

### Example 3-B

### Preparation of Amino Polyester Polyol (APPO)

The procedure of Example 1-B is used for this example. Thus from 100 parts of UPPO from Example 3-A, and 19.1 parts α-Methylbenzylamine a viscous yellow resin of amino polyester polyol (APPO) is obtained. The resultant light yellow viscous liquid obtained is filtered. The resulting resin has 97.6% non-volatile material (NVM); a Mn of 1300; a Mw of 2950; a hydroxyl equivalent weight of 938, an amine equivalent weight of 502, and an average equivalent weight of 327.

### Example 4

This example demonstrates the effect of steric hindrance of the primary amine used on speed of cure or gel time of APPO with isocyanate.

Example 3B is reproduced using equimolar amount of 2-methylcyclohexylamine instead of α-Methylbenzyl amine. Thus from 77.6 parts of unsaturated polyester from Example 3-A and 22.4 parts of 2-methylcyclohecylamine, a viscous light yellow liquid is obtained having a Mn of 1180, a Mw of 2780; a hydroxyl equivalent weight of 943, an amine equivalent weight of 506, and an average equivalent weight of 329.

### Example 5

This example demonstrates the effect of steric hindrance of the hydroxyl groups used on speed of cure or gel time of APPO with isocyanate.

The procedure of Example 3A is used here to prepare the hindered unsaturated polyesterpolyol. Thus from 52.6 parts of 2,2,4-trimethyl 1,3-pentanediol, 4.26 parts trimethylol propane, 9.7 parts adipic acid, 21.2 parts maleic anhydride, 20.8 parts isononanoic acid, and 0.2 parts triphenylphosphite, an unsaturated polyester polyol having an acid value of 7.8 is obtained. Glycildyl versatate (3 parts) is added to consume the residual acid and the reaction is heated at 125° C. for 6 hours. The resultant unsaturated polyester polyol (100 parts) is treated with α-methylbenzyl amine (25.4 parts) as described in Example 3-B. The resultant amino polyester polyol has an Mn of 1280, a Mw of 2880; a hydroxyl equivalent weight of 905, an amine equivalent weight of 597, and an average equivalent weight of 359.

### B) Coating Performance of Amino & Hydroxyl Functional Polyesters

White paints are made from resins made according to the present invention (Examples 6-12) and similar paints are made for Comparative Examples A and B. The following general procedure is used to make such white paints.

### General Procedure for the Preparation of White Paint:

To a high speed Cowles mixer, the following ingredients are added: 19.5 parts of APPO from Example 3-B, 2.0 parts xylene, 0.47 part of MPA 4020-X (anti settling agent from Elementis), 1.24 parts methyl amyl ketone (MAK), 1.41 parts and Disperbyk 163 (pigment dispersant from BYK-Chemie) and mixed for 5 minutes at low speed. Titanium dioxide (46.8 parts, R706 from DuPont) is sifted in slowly while mixing. Methyl amyl ketone (1.24 parts) is added and the slurry is dispersed for 15 minutes at high speed or until a Hegman grind of 6.5-7 is obtained. Additional resin from Example 3-B (9.76 parts), Byk 077 (0.47 part), Byk 306 (0.09 part) and methyl isobutyl ketone (4.31 parts) are added. The TiO₂ grind base is mixed for additional 20-30 minutes at low speed and 16.67 parts Desmodur N-3390 (from Bayer) is added.

### Comparative Examples A & B

Comparative Examples A & B are made according to the general paint procedures described above for using Desmophen NH-1520 (From Bayer) as the binder for Comparative Example A and acrylic polyol, 27-1316 (2.2% OH; from Nuplex Resins), as the binder for Comparative Example B.

### Example 6 to 10

### Coatings Performance of APPO

White paints are made from resins of Examples 1-B, 2-B, and 3-B, 4 and 5 as described in the general procedures above and various tests are carried out. Various tests, listed in Table 2, are carried out according to test methods and instruments listed in Table 1. The freshly prepared paints are used to measure the Kreb viscosity and gel time. For the latter test, about 100 g of a freshly activated white paint is placed in a paper cub and the L-shaped spindle of Shyodu gel Time (Model 100, made by Paul N. Gardner, Inc.) is immersed into the paint and start to rotate. Rotation continues until the paint is gelled and the spindle has stopped. The time required for the spindle to freely rotate is called the gel time. Paints are drawn down on Bonderite B-1000 panels with Doctor blade to give a 2-2.5 mils dry film thickness and the wet panels, immediately, placed under a Gardner dry-time recorder. Gloss, hardness, impact resistance, and conical Mandrel flexibility test are performed after drying at controlled temperature and humidity (72° F. and 50% RH, respectively).

TABLE 1
Equipment & ASTM Methods used for various Coatings Tests

| TEST | INSTRUMENT | ASTM METHOD No. |
|---|---|---|
| Viscosity | BYK Gardner | D-562 |
| | Model KU1 + Stormer | |
| 20°/60° Gloss | BYK-Gardner | D-523 |
| | Model 4430 Gloss Meter | |
| Drying Time | Gardco model DT-5020 | D-5895 |
| | Dry Timer | |
| Impact Resistance | Gardner Impact Tester | D-2794 |
| Elongation | Gardner Conical Mandrel | D-522 |
| Koenig | BYK-Gardner | D-5895 |
| Pendulum Hardness | Koenig Pendulum Tester | |

Coatings results of various tests are shown in Table 2 for Examples 8-12.

TABLE 2
Coatings Data for Examples 6-10

| | Exam- | Exam- | Exam- | Exam- | Exam- |
|---|---|---|---|---|---|
| | ple 6 | ple 7 | ple 8 | ple 9 | ple 10 |
| | Exam- | Exam- | Exam- | Exam- | Exam- |
| Resin Type | ple 1B | ple 2B | ple 3B | ple 4 | ple 5 |
| Paint Viscosity | 88 | 97 | 89 | 80 | 78 |
| (KU) | | | | | |
| Gloss 20°/60° | 87/93 | 93/97 | 89/95 | 85/93 | 89/95 |
| VOC, g/l | 218 | 218 | 218 | 227 | 227 |

Dry Times

| | | | | | |
|---|---|---|---|---|---|
| Dry touch | 18′ | 8′ | 16′ | 15′ | 16′ |
| Tack free | 54′ | 12′ | 34′ | 1 H 14′ | 1 H 10′ |
| Dry hard | 1 H 20′ | 22′ | 46′ | 2 H 36′ | 3 H 08′ |
| Dry through | 2 H 44′ | 38′ | 1 H 20′ | 4 H 14′ | 5 H 10′ |

KPH hardness

| | | | | | |
|---|---|---|---|---|---|
| 1 day | 10 | 47 | 14 | 33 | 22 |
| 7 day | 18 | 86 | 39 | 49 | 42 |

Impact Resistance

| | | | | | |
|---|---|---|---|---|---|
| 7 day (dir/rev) | 160/160 | 40/20 | 145/140 | 160/150 | 110/110 |
| inch. Lb. | | | | | |

Elongation (Conical Mandrel)

| | | | | | |
|---|---|---|---|---|---|
| 7 day | No Effect | No Effect | No Effect | No Effect | No Effect |
| Gel Times | 1 H, 6 min | 16 min | 41 min | 4 H 5′ | 4 H 35′ |

### Example 11 to 12 and Comparative Examples A & B

To test the advantages of the present invention, in comparison with the prior art, blends of white paints of Example 9 and those from Comparative Examples A or B, at 50-50% by weight blend ratio, yield Examples 12 and 13, respectively. Example 14 is a paint made from AAPO of Example 4. Blend of paint of Example 14 with a Comparative Example A provides Example 15. Coatings test data for Examples 12-15 and the Comparative Examples A and B are shown in Table 3.

TABLE 3
Coatings Data for Example 11-12 and Comparative Examples A & B:

| | Example No. | | | |
|---|---|---|---|---|
| | | | Comparative | Comparative |
| | 11 | 12 | Example A | Example B |
| Resin Type | 50/50% | 50/50 Blend | NH-1520* | 27-1316* |
| | Blend of | of | Aspartate | Acrylic Polyol |
| | Example 3 & | Example 3 & | | |
| | NH-1520 | 27-1316 | | |
| Paint | 74KU | 92 KU | 64 KU | 72 KU |
| Viscosity | | | | |
| Gloss | 88/93 | 89/94 | 87/93 | 89/95 |
| 20°/60° | | | | |
| VOC, g/l | 197 | 226 | 215 | 333 |

Dry times

| | | | | |
|---|---|---|---|---|
| Dry touch | 16′ | 18′ | 1H 30′ | 30′ |
| Tack free | 1H | 32′ | 2H 40′ | 2H 45′ |
| Dry hard | 1H 40′ | 1H | 5H 50′ | 13H 30′ |
| Dry through | 2H 50′ | 2H 15′ | 10H | >24H |

KPH hardness

| | | | | |
|---|---|---|---|---|
| 1 day | 81 | 14 | 149 | 14 |
| 7 day | 129 | 37 | 176 | 54 |

Impact Resistance

| | | | | |
|---|---|---|---|---|
| 7 day | 45/20 | 120/135 | 10/0 | 130/130 |
| (dir/rev) | | | | |
| inch. lb. | | | | |

Elongation (Conical Mandrel)

| | | | | |
|---|---|---|---|---|
| 7 day | No Effect | No Effect | Complete | No Effect |
| | | | delamination | |
| Gel Time | 2H, 24′ | 1H 47′ | 11H, 8′ | >24H |

*Comparative Example A = NH-1520 a Polyaspartate Resin from Bayer
**Comparative Example B = 27-1316 an Acrylic Polyol (2.2% OH) from Nuplex Resins

Results of Tables 1 and 2 clearly demonstrate that coatings containing resins of the present invention have shorter dry-times, higher impact resistance and better elongation or flexibility than coatings based on the prior art shown in the Comparative Experiments A and B.

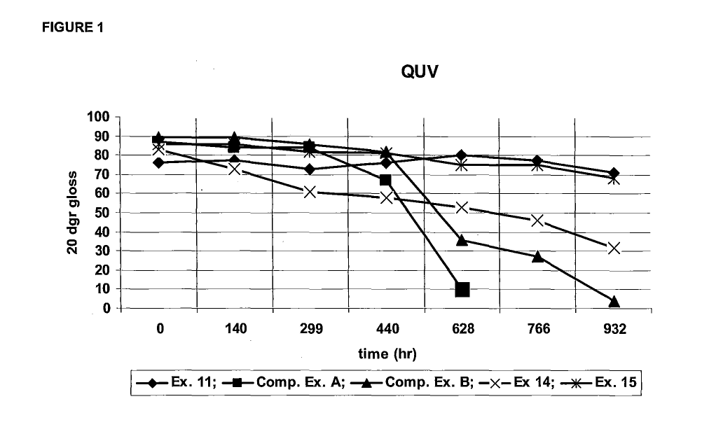

The QUV 313 accelerated weathering of white paints of Examples 8, 11, 12, and Comparative Examples A and B are shown in FIG. 1 which shows the 20° Gloss Retention of several amino and hydroxy-Functional compounds in white paint as described in several of the examples.

QUV is the name of the instrument made by the Q-Lab company. A QUV test chamber uses fluorescent lamps to provide a radiation spectrum centered in the ultraviolet wavelengths. Moisture is provided by forced condensation, and temperature is controlled by heaters. The test references that can be referred to are: ASTM D4329, D4587ASTM D4329, D4587.

The results of the FIGURE clearly demonstrate the superior durability of the coatings based on the present invention over those of the prior art. In addition, it shows that paint made according to the present invention can improve the durability of conventional acrylic urethane coatings (Example 12).

### C) The Following Examples Illustrate the Special Features of the Low Molecular Weight Amino and Hydroxy-Functional Polyesters

### Example 13-A

### Preparation of Monobutyl Maleate

A four-necked reaction flask equipped with a condenser, agitator, heating mantle; addition funnel, thermocouple attached to a temperature control, and Dean-Stark trap primed with xylene, is charged with 57.0 parts (by weight) of maleic anhydride and 43.0 parts (by weight) of 1-butanol and heated under 0.5 SCFH (standard cubic feet per hour) (0.014 m³ hr⁻¹) nitrogen flow to 50° C. The progress of the reaction was monitored by fourier transform infrared spectroscopy (FT-IR). The reaction was carried out at this temperature until the disappearance of anhydride absorption at 1776 and 1851 cm⁻¹. The obtained monobutyl maleate is stored in a glass reactor. The unsaturated monobutyl maleate is filtered and stored. It has a non-volatile material of 100%; an acid value of 326; an unsaturated equivalent wt. of 172; a number average molecular weight (Mn) of 262; a weight average molecular weight (Mw) of 271; and a polydispersity of 1.03.

### Example 13-B

### Preparation of 100% NVM Unsaturated Polyester Polyols

A four-neck reaction flask equipped as in Example 13-A, is charged with 57 parts (by weight) of oxirane-2-ylmethyl 2,2-dimethyloctanoate (Cardura-E-10). N,N,dimethylbenzyl amine (1 g) was added as a catalyst. Monobutyl maleate (43 parts by weight of Example 13-A) was added slowly over two hours. After the addition, the reaction was heated under 0.5 SCFH (standard cubic feet per hour) (0.014 m³ hr⁻¹) nitrogen flow to 125° C. and maintained at such temperature until an acid value of less than 2 is attained. No solvent was added to the reaction. The unsaturated polyester polyol is filtered and stored. The obtained unsaturated polyester polyol has a non-volatile material of 100% (measured by mixing the resin with NCO at NCO/OH of 1:1 and heated for 1 hr. at 130° C.); an acid value of 1.6; a hydroxyl value of 140; an unsaturated equivalent wt. of 400; a number average molecular weight (Mn) of 469; a weight average molecular weight (Mw) of 565; and a polydispersity of 1.2.

### Example 13-C

### Preparation of Amino Polyester Polyol

A four-neck reaction flask equipped as in Example 13-A, is charged with 84.6 parts of Example 13-B. Sec-butyl amine (15.4 parts) is added drop-wise via addition funnel over 1 hour while maintaining the temperature at or below 50° C. Heating is continued overnight at 50° C. The resultant light yellow viscous liquid obtained is filtered and stored at room temperature for 6 weeks. The resulting resin has a non-volatile material (NVM) of 100%; a viscosity of 1100 m Pa·s; a number average molecular weight (Mn) of 524; a weight average molecular weight (Mw) of 654; and a polydispersity of 1.25. The hydroxyl equivalent weight is 467 (OH value=120), and the amine equivalent weight is 474 (amine value=118) and the average equivalent weight for the total functionality is 470.5. The amine value is determined by titrating the amine with 0.1 N hydrochloric acid (ASTM method D2572-91). The total hydroxyl plus the amine value is determined by acetylating both the hydroxy and amine groups with acetic acid anhydride and then titrating acetic acid with KOH. The hydroxyl value is then determined by subtracting the amine value from the total amine plus hydroxyl values.

### Example 14-A

### Preparation of 100% NVM Unsaturated Polyester Polyols

A four-necked reaction flask equipped with a condenser, agitator, heating mantle; addition funnel, thermocouple attached to a temperature control, and Dean-Stark trap primed with xylene, is charged with 51.6 parts (by weight) of bis(3,4-epoxycyclohexylmethyl) adipate (ERL 4299; a commercial product from Dow Chemicals). N,N,dimethylbenzyl amine (1 g) was added as a catalyst. Monobutyl maleate (48.4 part by weight of Example 1-A) was added slowly over two hours. After the addition, the reaction was heated under 0.5 SCFH (standard cubic feet per hour) (0.014 m³ hr⁻¹) nitrogen flow to 125° C. and maintained at such temperature until an acid value of less than 2 is attained. No solvent was added to the reaction. The unsaturated polyester polyol is filtered and stored. The obtained resins has a non-volatile material of 100% (measured by mixing the resin with NCO at NCO/OH of 1:1 and heated for 1 hr. at 130° C.); an acid value of 1.6; a hydroxyl value of 158; an unsaturated equivalent wt. of 355.4; a number average molecular weight (Mn) of 1100; a weight average molecular weight (Mw) of 3000; and a polydispersity of 2.72.

### Example 14-B

### Preparation of Amino Polyester Polyol

A four-neck reaction flask equipped as in Example 13-B, is charged with 82.9 parts of unsaturated polyester polyols from Example 14-B. Sec-butyl amine (17.1 parts) is added drop-wise via addition funnel over 1 hour while maintaining the temperature at or below 50° C. Heating is continued overnight at 50° C. The resultant light yellow viscous liquid obtained is filtered and stored for 6 weeks at room temperature. The resulting resin has a non-volatile material (NVM) of 100%; a number average molecular weight (Mn) of 1150; a weight average molecular weight (Mw) of 3100; and a polydispersity of 2.70. The hydroxyl equivalent weight is 428 (OH value=131), and the amine equivalent weight is 428.5 (amine value=131) and the average equivalent weight for the total functionality is 428. The amine value is determined by titrating the amine with 0.1 N hydrochloric acid (ASTM method D2572-91). The total hydroxyl plus the amine value is determined by acetylating both the hydroxy and amine groups with acetic acid anhydride and then titrating acetic acid with KOH. The hydroxyl value is then determined by subtracting the amine value from the total amine plus hydroxyl values.

### Example 15

Example 15 is an example of for the preparation of polyester with on average 1.9 hydroxyl groups and 1 NH group per molecule.

### Example 15-A

### Preparation of Unsaturated Polyester Polyols

A four-necked reaction flask equipped with a condenser, agitator, heating mantle; addition funnel, thermocouple attached to a temperature control, and Dean-Stark trap primed with xylene, is charged with 74.22 parts (by weight) of 2,2,4-trimethyl-1,3-pentanediol (TMPD), 25.78 parts of and, 0.035 part of Fascat 9100 (butyl stanoic acid catalyst available from Arkema Inc.), and heated under 0.5 SCFH (standard cubic feet per hour) (0.014 m³ hr⁻¹) nitrogen flow to 195-200° C. At 165° C., water starts to distil azeotropically. The reaction temperature is gradually increased to 200° C. and maintained at such temperature until an acid value of less than 2 is attained. The azeotropic water collected is 5.2 parts. The Dean-Stark trap is drained and the reaction mixture is purged with nitrogen to remove most of the volatiles. The unsaturated polyester polyol is cooled to 150° C. and filtered. The obtained resin has a non-volatile material of 98% (measured by mixing the resin with NCO at NCO/OH of 1:1 and heated for 1 hr. at 130° C.); an acid value of 1.31; a hydroxyl value of 569.6; a number average molecular weight (Mn) of 700; a weight average molecular weight (Mw) of 1050; and a polydispersity of 1.50.

### Example 15-B

### Preparation of Amino Polyester Polyol

A four-neck reaction flask equipped as in Example 15-A, is charged with 83.7 parts of unsaturated polyester polyols from Example 15-A. Sec-butylamine (16.3 parts) is added drop-wise via addition funnel over 1 hour while maintaining the temperature at or below 50° C. Heating is continued overnight at 50° C. The resultant light yellow viscous liquid obtained is filtered. The resulting resin has a non-volatile material (NVM) of 98.3%; a viscosity of 40000 m Pa·s; a number average molecular weight (Mn) of 695; a weight average molecular weight (Mw) of 1047; and a polydispersity of 1.5. The hydroxyl equivalent weight is 235 (% OH=7.25; OH value=238), and the amine equivalent weight is 436 (% N=3.2; amine value=129) and the average equivalent weight for the total functionality is 153. The amine value is determined by titrating the amine with 0.1 N hydrochloric acid (ASTM method D2572-91). The total hydroxyl plus the amine value is determined by acetylating both the hydroxy and amine groups with acetic acid anhydride and then titrating acetic acid with KOH. The hydroxyl value is then determined by subtracting the amine value from the total amine plus hydroxyl values.

### Example 16

Example 16 is the same as Example 14 except that the primary amine used is tertiarybutyl amine instead of secondary butyl amine. The resulting resin has a non-volatile material (NVM) of 99.0%; an average molecular weight (Mn) of 760; a weight average molecular weight (Mw) of 1140; and a polydispersity of 1.5. The hydroxyl equivalent weight is 235 (OH value=238), and the amine equivalent weight is 436 (amine value=129) and the average equivalent weight for the total functionality is 153.

### Comparative Example C

This example is made according to the prior art. Thus from a procedure similar to that described above, the following materials were processed to make the unsaturated polyester: Neoppentyl glycol (34.65 parts); trimethylol propane (4.4 parts), isononanoic acid (21 parts); 1,6-hexanediol (17.6 parts) and maleic anhydride (30.3 parts) there were obtained unsaturated polyester polyol with hydroxyl equivalent weight of 323 (% OH of 5.25); and unsaturation equivalent weight of 323 which corresponds to an average of 3 unsaturated units per molecule. The acid value was 2.1 and % solids of 96.65%. This unsaturated polyester was treated with 2-methylcyclohexylamine to yield amino polyester polyol The resulting resin has a non-volatile material (NVM) of 97.3%; an average molecular weight (Mn) of 1000; a weight average molecular weight (Mw) of 2100; and a polydispersity of 2.1. The hydroxyl equivalent weight is 436 (% OH of 3.9; OH value of 129), and the amine equivalent weight is 436 (% nitrogen 3.2; amine value=129) and the average equivalent weight for the total functionality is 218. This corresponds to 2.5 groups of OH and 2.5 groups of NH per molecule.

Table 4 shows the hardness development of white pigmented coatings, made from Examples 15 &16 and from the Comparative Example C, over several weeks as measured by a Köning Pendulum Hardness tester. It can be easily observed that the hardness of coatings made from the prior art, Comparative Example C, shows a hardness increase up to 1-2 weeks and then the hardness decreases with time. While the hardness of the present inventions, Examples 15 & 16, show an increase to reach a maximum value and then stay constant with time.

TABLE 4
Hardness Measurements of Pigmented Coatings with Time

| Time | Comparative | Time | Example | Example |
|---|---|---|---|---|
| (day) | Example C | (day) | 16 | 15 |
| 1 | 48 | 1 | 52 | 25 |
| 4 | 113 | 2 | 81 | 76 |
| 7 | 126 | 3 | 90 | 124 |
| 14 | 114 | 7 | 90 | 180 |
| 27 | 90 | 14 | 87 | 190 |
| 43 | 63 | 21 | 85 | 194 |
| | | 77 | 88 | 187 |

Table 5 shows the clear coating data for Example 13, 15 & 16. The physical properties such as hardness, impact, and flexibility can be tailored to the desired levels with the use of various resins of present invention.

TABLE 5
Coatings Data for Examples: 13, 15, & 16

| Resin Type | Example 13 | Example 15 | Example 16 |
|---|---|---|---|
| Clear coat % Solids | 74% | 75% | 75% |
| % DBTDL | 0.03 | 0.04% | 0-.01% |

Dry Times

| | | | |
|---|---|---|---|
| Dry touch | 1 H | 1 H | 35 min |
| Tack free | 3 H | 2 H | 2 H |
| Dry hard | 12 H | 6 H | 9 H |

KPH Hardness

| | | | |
|---|---|---|---|
| 1 day | 24 | 52 | 25 |
| 7 day | 35 | 90 | 180 |
| 76 day | | 88 | 187 |

Impact Resistance

| | | | |
|---|---|---|---|
| 7 day (dir/rev) inch. Lb. | 160/160 | 85/60 | 45/30 |

Elongation (Conical Mandrel)

| | | | |
|---|---|---|---|
| 7 day | No Effect | No Effect | No Effect |
| Gel Times | 45 H, 43 min | 45 H, 43 min | 4 H 27 min |

### Examples 17 & 18

### Blends of Amino and Hydroxy-Functional Polyesters with Acrylic Polyols

Table 6 shows the coatings properties of white paints were made from 50/50 blends by weight of Example 13 and 16 with an acrylic polyol 27-1316 (2.2% OH; a commercial product from Nuplex Resins). The hardness and VOC are dramatically improved for the blends.

TABLE 6
Coatings Data for Examples: 17 & 18

| | | | Comparative |
|---|---|---|---|
| Example No. | Example 17 | Example 18 | Example B |
| Resin Type | 50/50 Blend of | 50/50 Blend of | 27-1316 |
| | Example 13 & | Example 16 & | Acrylic Polyol |
| | 27-1316 | 27-1316 | |
| % Solids | 78% | 76% | 76% |
| % DBTDL | 0.03 | 0.04% | 0.001% |
| Oxsol-100 needed for | 11.8% | 14.9% | VOC = 333 g/l |
| 100 g/l VOC | | | |

Dry Times

| | | | |
|---|---|---|---|
| Dry touch | 45 min | 50 min | 30 min |
| Tack free | 2 H | 4 H 30 min | 2 H 45 min |
| Dry hard | 10 H 20 min | 18 H | 13 H 30 min |

KPH Hardness

| | | | |
|---|---|---|---|
| 1 day | 19 | 18 | 14 |
| 2 day | 29 | 49 | |
| 5 day | 43 | 107 | 7 days = 54 |
| Gel Times | 2 H, 17 min | 19 H, 36 min | >24 H |

Further modifications in addition to those described above may be made to the structures and techniques described herein without departing from the spirit and scope of the invention. Accordingly, although specific embodiments have been described, these are examples only and are not limiting upon the scope of the invention.

What is claimed is:

1. An amino and hydroxy-functional polyester, wherein the amine is in the form of aspartic acid esters functionality, and wherein the amino and hydroxy-functional polyester has
(a) a molecular weight (Mn) of at least about 500;
(b) an acid value of about 5 or less;
(c) a hydroxyl value of about 30 or more; and
(d) an amine value of about 30 or more
(e) and wherein the hydroxyl is a sterically-hindered primary and/or secondary hydroxy.

2. The amino and hydroxy-functional polyester according to claim 1, wherein molecules of the polyester have on the average:
(f) at least 1 secondary amino group as an aspartate,
(g) and/or at least 1 hydroxy group,
(h) and an average total functionality of about 1.8 or higher.

3. The amino and hydroxy-functional compound according to claim 1, wherein the compound is in the form of a polyester polyol, and wherein the polyester has a general structure according to formula I: 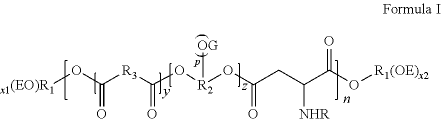
wherein: R=mono-valent alkyl, aryl and/or arylalkyl radical;
R₁=residue obtained from a polyol after removing OH groups and wherein the R₁ has a valency of 1 to 6;
R₂=residue obtained from a polyol after removing OH groups and wherein the R₂ has a valency of 2 to 6;
R₃=divalent saturated and/or unsaturated alkyl and/or aryl radical;
E=H or acyl group having 1 to 18 carbon atoms;
X¹, X²=an integer having an equal or different values of 0 to 5, and wherein a sum of X¹ and X² is at least 1;
y, z=an integer having a value of 0 or 1;
p=an integer having a value between 0 to 4;
G=E and/or is a residue having the following structure: 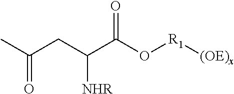
n=an integer having a value between 1 to 10.

4. The amino and hydroxy-functional polyester according to claim 1, wherein the number average molecular weight of the amino and hydroxy-functional polyester is about 500 or higher and about 5,000 or lower.

5. The amino and hydroxy-functional polyester according to claim 1, wherein the polydispersity of the amino and hydroxy-functional compound is about 4 or lower, and about 1.2 or higher.

6. The amino and hydroxy-functional polyester according to claim 1, wherein the amino and hydroxy-functional compound has a hydroxyl value of about 40 or higher and of about 300 or lower.

7. The amino and hydroxy-functional polyester according to claim 1, wherein the amino and hydroxy-functional polymer has an amine value of about 40 or higher and of about 300 or lower.

8. The amino and hydroxy-functional polyester according to claim 1, wherein the amino and hydroxy-functional polymer has an average total functionality of about 1.8 or more and of about 10 or less.

9. A curable composition comprising: the amino and hydroxy-functional polyester according to claim 1 and a polyisocyanate, wherein the amount of isocyanate is present in about 60% of the molar amount of the amino and alcohol groups, or more.

10. A method for preparing a polyester based amino and hydroxy-functional compound, comprising the preparing a hydroxyl functional polyester comprising at least one of: maleate and fumarate unsaturation, and wherein the maleate and fumarate unsaturation are reacted with an aliphatic or aromatic amine compound to prepare an aspartate through a pseudo Michael addition reaction.

11. A coating composition comprising the following components in parts by weight (pbw):
(a) amino and hydroxy-functional polyester (1-80 pbw); wherein the amine is in the form of aspartic acid esters functionality, and wherein the amino and hydroxy-functional polyester has:
(i) a molecular weight (Mn) of at least about 500;
(ii) an acid value of about 5 or less;
(iii) a hydroxyl value of about 30 or more; and
(iv) an amine value of about 30 or more
(v) and wherein the hydroxyl is a sterically-hindered primary and/or secondary hydroxy;
(b) polyisocyanate compound (1-65 pbw)
(c) other binder constituents (0-60 pbw)
(d) colorants (0-40 pbw)
(e) additives (0-10 pbw)
(f) tin catalyst (0-0.1 pbw)
(g) solvents (0-30 pbw)
wherein components (a)-(f) together are 100 pbw.

12. The composition according to claim 11, and wherein component (c) comprises one or more of:
(i) hydroxy functional acrylic polymers, hydroxy functional polyester, hydroxy functional reactive diluent, hydroxy functional polyether, hydroxy functional polycarbonate or hydroxy functional polyurethane;
(ii) non-functional polymers or functional polymers with a functionality equivalent weight of about 5000 or higher; and
(iii) aspartate functional compounds other than compound (a).

13. The composition according to claim 11, wherein component (c) is present in an amount between 5 and 50 pbw.

14. The composition according to claim 11, wherein component (c) comprises a hydroxy functional acrylic polymer.

15. The composition according to claim 11, wherein component (c) comprises an aspartate functional compound other than component (a).

16. The composition according to claim 11, wherein component (g) is present in about 10 pbw relative to components (a) through (f) or less.

17. A low molecular weight amino and hydroxy-functional polyester derived from the reaction of unsaturated oligoesters and mono primary amine, the unsaturated polyesters having on average at least one and a maximum of 1.8 fumarate or maleate units, and wherein the amino functionality is between 0.8 and 1.8 groups per molecule in the form of a secondary amine as aspartic acid ester.

18. The polyester of claim 17, wherein the hydroxyl functionality is between 1 and 12 hydroxyl groups per molecule.

19. The polyester according to claim 17, wherein the molecular weight of the amino and hydroxy-functional polyesters is between 204 and 10,000.

20. The polyester according to claim 17, wherein the amino and hydroxy-functional compounds contain 0.1 to 7% by weight nitrogen in the form of secondary amine as aspartate groups and 0.1% to 10% of primary and/or secondary hydroxyl groups.

21. The polyester according to claim 17, wherein the general structure of the amino and hydroxy-functional polyesters is shown in Formula II 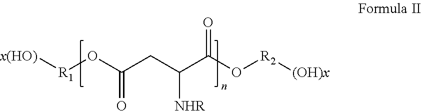
wherein: R=mono-valent alkyl, aryl and/or arylalkyl radical and optionally contains an OH group; and
R₁ and R₂ each is a residue obtained from a polyol after removing the OH groups and having a valency of 1 to 6, and x=is an integer having a value between 0 to 6, and n has a value between 1 to 1.8.
